(19) United States
(12) Patent Application Publication
(10) Pub. No.: US 2007/0172900 A1
(43) Pub. Date: Jul. 26, 2007

(54) Diagnostic marker for cancer

(75) Inventors: Michael Cahill, Lorzweiler (DE); Helmut Klocker, Inzing (AT); Herman Rogatsch, Klagenfurt (AT)

Correspondence Address: NATH & ASSOCIATES, 112 South West Street, Alexandria, VA, 22314, US

(73) Assignee: PROTEOSYS AG, Mainz (DE)

(21) Appl. No.: 10/589,487

(22) PCT Filed: Feb. 16, 2005
(86) PCT No.: PCT/EP05/01567

§ 371 Date: Nov. 9, 2006

(30) Foreign Application Priority Data
Feb. 16, 2004 (DE) 10 2004 008 449.1
Jul. 29, 2004 (DE) 10 2004 038 076.7

(51) Int. Cl. G01N 33/574; A61K 39/395

(52) U.S. Cl. 435/7.23; 424/155.1

(57) ABSTRACT

The invention relates to the use of various proteins as diagnostic markers for cancerous diseases. In particular, the use of the annexin A3 protein is preferred. Preferably an increased regulation of annexin A3 is analysed in comparison to controls. The invention also relates to the use of active substances for producing a medicament used in the treatment of cancer, said substances influencing the activity and/or abundance of various characteristic proteins.

The present invention relates to the use of a variety of different proteins as diagnostic markers for cancer, the application of active substances for the treatment of cancer and related pharmaceutical preparations and kits.

Generally cancerous diseases are characterized by the development of one or more tumours. Tumour means tissue growth or local increase of tissue volume. In a broader sense every localized swelling falls into this classification, e.g. oedema, acute or chronic inflammation, dilatations caused by aneurisms, inflammatic growth of organs (e.g. a so-called spleen tumour). In a more narrow sense a tumour implies formation of novel tissue (excrescence, blastoma, neoplasia) by spontaneous, uncontrolled, uninhibited to diverse grades, autonomous and irreversible growth of body tissue in connection with loss of specific functions of cells and tissue.

For a better classification tumours are divided according to:

I. Their biological properties

1. Benign tumours with differentiated cells growing slowly and displacing normal tissue
2. Malignant tumours showing polymorphism of cell nuclei, atypical cells, anaplasia, invasive and destructive growth and metastasis.
3. Semi-malignant tumours displaying histological characteristics of malignant tumours and locally infiltrating growth but usually lacking metastasis.
   II. Histogenetic criteria:
In this connection tumours are classified according to their embryonic tissue of development origin. There are
1. Epithelial tumours, which have originated from ectoderm or endoderm:
a) benign tumours like adenoma, papilloma or polyps
b) malignant tumours e.g. carcinoma
2. Mesenchymal tumours, originating from mesoderm:
a) benign tumours like lipoma, fibroma, osteoma, myoma, leiomyoma, rhabdomyoma, chondroma
b) malignant tumours e.g. sarcomas
3. Embryonic tumours have developed from undifferentiated tissue. Nephroblastoma, neuroblastoma, medulloblastoma, retinoblastoma, embryonic rhabdomyosarcoma and teratoma are belonging to this section.
   III. Classification according to clinical and pathological results. Among these are TNM-Classification, Grading, Lauren-Classification, Dukes-Classification, Kieler Classification, Rappaport-Classification etc.

Even this short survey of tumour classification shows how diverse, overlapping and even contradictory the different types of tumours are. There is not only the difference between benign and malignant tumours; the mortality of certain tumours must be considered as well as the probability that a benign tumour can become malignant.

Some tumours, e.g. mamma carcinoma (breast cancer), the most common malignant tumours in women, occur in large numbers especially between age 45 and 70. Early symptoms are suspicious results of examinations in the framework of cancer prophylaxis or during regular self examination of the breast. Depending on the stage of tumour development and differentiation prognosis can range from very positive to really bad. Because of early lymphogenic and haematogenic metastasis of breast cancer, rapid diagnostics is essential to start therapeutic measures as soon as possible.

Prostate cancer (carcinoma of the prostate gland) is the most common tumour in men, occurring mostly between age 50 and 70. The majority of cases are adenocarcinomas. This type of malignant tumour first spreads through invasive growth inside the prostate gland and later infiltrates cells in the transition zone and connective tissues of pelvis and rarely intestines, bladder or urethra. Metastasis takes place via lymphogenic and/or haematogenic pathways. Therapeutic measures depend on histological grade of differentiation and clinical stage, and usually imply radical surgery, thus completely removing the prostate gland and regional of lymph noses; in progressive stages withdrawal of male sex hormones is a measure. Prognosis also depends on the staging of the tumours. Radical surgery in early stages cures about 90% of prostate cancers, while prognosis is often poor in progressive stages.

Prostate cancers have to be differentiated from prostate hyperplasia by diagnostic means. Prostate hyperplasia is a benign tumour of the prostate gland; the gland becomes enlarged by numerical increase of stroma cells and glands. Prostate hyperplasia is the most common cause for urination difficulties in elderly men. Clinical symptoms usually occur between age 40 and 50 and the disease progresses slowly and stepwise. Symptoms often appear years later with gradual weakening of the urine jet and delayed micturition. Application of phytotherapeuticals may be of therapeutic use and relieve clinical symptoms.

In general early diagnosis of tumours is essential for rapid start of therapeutic measures. Prognosis is improved if the tumour is detected early; therefore a number of so-called tumour markers are used in clinical practice. Tumour markers are molecules or cellular alterations which can be identified and quantified in order to gain information about existence, progression and prognosis of (malignant) disorders. Tumour markers are divided into:

1. Cellular Tumour Markers

This group contains among others tumour antigens of the cell membrane, receptors (e.g. hormone receptors, receptors for growth stimulating substances in leukaemia) and cellular markers which represent increased expression of cancer genes and monoclonal cell growth as well as genetic alterations, especially chromosomal aberrations.

2. Humoral Tumour Markers

Under physiological conditions increased concentrations of these substances (which mostly belong to the normal physiological repertoire) can be detected in biological samples, especially in serum, urine and other body fluids. They are synthesized and/or secreted in tumour tissues, released by disrupted tumour cells or in response of the organism to the tumour. The physiological relevance of tumour markers is only poorly understood. In the human body they normally do not have immunogenic properties. Their clinical (diagnostic) significance depends on specificity and sensitivity. Humoral tumour markers are grouped in two classes. One group includes markers produced by the tumour, e.g. tumour associated antigens, special hormones (e.g. Gastrin, Cortisol etc.), enzymes (e.g. neuron-specific enolase, NSE) or proteins (e.g. Bence-Jones-protein). Tumour markers induced by the tumour but not produced by the tumour cells belong to the second group. Important members of the second group are alkaline phosphatase (AP), LDH (lactate dehydrogenase), neopterin, etc.

Recently publication took place of a list of proteins, which could be detected in two representative cell lines of the medulloblastoma, the most frequent brain tumour in children and which can possibly be used as tumour markers (A. Peyrl et al., 2003, Proteomics, 3, 1781-1800). U.S. Pat. No. 6,645,465 discloses that annexins A1 and A2 belonging to the Ca²⁺ binding proteins can be used as tumour markers for lung, breast and oesophageal cancer and can be identified by detection of auto-antibodies directed against them. It was possible to show in animal tests that the use of radioactively labelled antibodies against annexin A1 leads to a tumour mass loss, which can probably be attributed to the cytoclasis of the tumour cells (P. Oh, Y. Li, J. Yu, E. Durr, R. M. Krasinska, L. A. Carver, J. E. Testa, J. E. Schnitzer, 2004, Nature, 429, 629-35).

Recently a differential abundance analysis has been performed in malignant and non-malignant (benign) pancreatic epithelial cells Annexin A3 being indicated as an identified protein in this connection (A. R. Shekouh et al., 2003, Proteomics, 3, 1988-2001). The abundance of protein in malignant and non-malignant prostate tissue has also been investigated. With regards to the proteins identified in this connection, largely no further details have been given regarding a possible overexpression or underexpression of the listed proteins in cancerous tissue compared with healthy tissue (A. A. Alaiya et al., 2001, Cell. Mol. Life Sci., 58, 307-311).

The statements made until now demonstrate the importance of selective and sensitive methods for tumour detection. Moreover there is great demand for new targets for tumour and cancer therapy respectively.

Therefore this invention relates to the problem of developing new markers for cancer diagnosis and new targets and drugs for cancer therapy.

This problem will be solved by the subjects of the independent claims. Preferential embodiments are given in the dependent claims. By reference the wording of the complete claims becomes an integral part of the description.

By means of intensive comparative analyses between malignantly degenerate tissue (cancer tissue) and normal tissue, a distinct set of proteins could be identified that showed significantly different abundance or concentration in the different types of tissue. The characteristic abundance of a certain protein compared to controls represents an important indication for degenerate cell growth, i.e. cancer tissue. According to the invention these particular proteins are used as diagnostic markers for cancer.

In order to identify these proteins, samples from tumour tissue (prostate cancer) and healthy tissue were prepared and the two samples were labelled with two different radioactive isotopes. The samples were pooled and the mixture was separated by electrophoresis on a two-dimensional polyacrylamide gel. The signals of every isotope were detected individually and the corresponding protein spots were further analyzed. This method identified and quantified several distinct proteins with significantly different abundance in cancer or healthy tissue. Some of these proteins are significantly more abundant in cancer tissue, they are upregulated, and others occur with significantly lower abundance, they are downregulated.

The invention covers the application of the protein annexin, especially annexin A3, as a diagnostic marker for cancer. The inventors were able to demonstrate, that this protein is upregulated on average 2.4 times and in certain cases more than 5 times in tumour tissue from patients of defined collectives. In a particularly preferred embodiment annexin A3 can thus be used as a diagnostic marker for specific subtypes (patient groups) of prostate cancer. Therefore upregulation of this protein compare to controls is preferentially studied as characteristic indicator for cancer tissue. The annexins are members of a family of structurally related proteins which bind phospholipids in the presence of calcium and form calcium pores. Until now the precise function of the annexins is still not completely clear.

There are indications, that annexins take part in intracellular and extracellular processes. But it is not known how annexins are secreted e.g. membrane trafficking, cell mobility Ca²⁺ influx and signal transduction. For example they have no classical leader sequences for translation into the lumen of the endoplasmic reticulum. But annexins can be found in small secrete vesicles, so-called exosomes therefore it is suspected that annexins reach the outside of the cell via lysis of the exosomes. Lysis of said vesicles can lead to a modified antigen presentation in tumours. Generally exosomes are involved in antigen presentation in the immune system, they are related to the MHC class I/T-cell system.

Interestingly annexins take part in bone mineralization (Wang W. Xu J., Kirsch T 2003 J. Biol. Chem. 2003, 278: 3762-9). Annexin-mediated Ca²⁺ influx regulate growth plate chondrocyte maturation and apoptosis. This is especially remarkable because metastases of prostate cancer produce an unusually high frequency of osteoblastic bone lesions compared to other types of cancer. Most cancer metastases are characterized by their osteolytic activity, which means degeneration of bones. In contrast prostate cancer metastases show osteoclastic (destructive) as well as osteoblastic (proliferative) activity. In this case normal bone crystals are deconstructed and then built up again as disordered bone deposit. Even though this mechanism is only poorly understood, physiological processes of mineralization play an important role. Mineralization is initiated by small vesicles, so-called matrix vesicles, which are secreted by the plasma membranes of mineralizing osteoblasts. In early stages crystal of calcium phosphate are emerging inside the matrix vesicles. These vesicles are covered by membranes; therefore channel proteins are necessary to transport minerals into the vesicles. Important components of the vesicles are the proteins annexins A2, A5 and A6 as well as collagen type II and X on the outer surface of the vesicles which bind to annexin A5 to adhere to the outer surface of the vesicles. Annexins form channels through the membranes of the matrix vesicles which allow Ca²⁺ to enter the vesicles interior. Collagen bound to annexin A5 amplifies the channel activity and mediates together with other annexins the rapid influx of Ca²⁺ and the formation of the first crystalline phase inside the vesicles. This results in the initiation of mineralization. When the intracellular crystals have reached a critical size they destroy the membrane and lyse the vesicles. The crystals grow further (growth stage of mineralization) and contribute to the building of bones. According to the inventors' results this function of the annexins in the irregular mineralization of bones by prostate cancer metastases is presumably linked to the upregulation of annexin A3 in cancer tissue. In this context inorganic pyrophosphatase 1 should be taken into account this enzyme releases phosphate and is upregulated in cancer, especially prostate cancer, according to the inventors' results. From upregulation of annexin A3 in cancer cells one has to conclude that annexin A3 has a biological function in the exosomes of prostate cancer cells. This is possibly due to a relation to ion channels. A preferred application of protein annexin A3 relates to the activity of the protein in exosomes. Preferentially this leads to changes in the immunologic control of tumour cells. Because the extracellular concentration of annexin A3 is higher in the vicinity of tumour cells an affinity reagent—especially an antibody with high affinity for annexin A3—will be suitable to direct active substances like toxin or radioactive compounds near the tumour. Such a medicament should not pass through the cell membrane so that healthy cells which express only intracellular annexin A3 are not affected. Interestingly matrix vesicles have also been observe in connection with osteoarthritic cartilage an atherosclerotic lesion.

The release of cytoplasmic proteins into the extracellular medium taking place following lysis of exosomes can induce an inflammatory response which is similar to that with cell necrosis. It is known that an inflammation can reduce the adaptive T-cell-caused immune response known to characterize many cancer cells. In addition, the presence of annexins in extracellular space can also influence this pattern (A. Bonanza et al., 2004 J. Exp. Med. 200 1157-65). Therefore a vaccination against cancer can be determined by understanding and influencing this system.

A particularly preferred embodiment of application of annexin A3 relates to the upregulation of the protein and simultaneous downregulation of annexin A1, annexin A2 an/or annexin A5. Preferentially this will be done in comparison with controls. It has been demonstrated recently that annexin A1 annexin A2 and annexin A5 are downregulated in cancerous tissue, especially in prostate cancer. Therefore analyzing upregulation of annexin A3 together with downregulation of one or more other annexins will be particularly informative. On the basis of these results annexin A3 could replace other annexins during prostate carcinogenesis and therefore be a replacement marker or target for prostate cancer treatment.

The invention also covers the application of the proteins ubiquitin isopeptidase T and/or protein disulphide isomerase (PDI) as diagnostic markers for cancer. Advantageously downregulation of ubiquitin isopeptidase T and/or upregulation of protein disulphide isomerase (PDI) compared to controls should be used as characteristic markers for cancerous tissue. The inventors were able to demonstrate that ubiquitin isopeptidase T is about 5 times less abundant and PDI about twice as abundant in tumour tissue compared to healthy tissue. This demonstrates an inverse correlation between PDI and ubiquitin isopeptidase T.

Ubiquitin isopeptidase is an enzyme which—among other enzymes—is involved in ubiquitin-dependent proteolytic cleavage of proteins. After addition of a polyubiquitin chain to the target protein the ubiquitinylated protein will be degraded by the 26 S proteasome; a protein complex consisting of many subunits. Subsequently removal of the polyubiquitin chain is mediated by the zinc-binding ubiquitin enzyme isopeptidase T. The downregulatien of ubiquitin isopeptidase T could therefore influence the speed of ubiquitin-cause proteolysis in prostate cancer or the degradation rate of specific proteins. As well as ubiquitin isopeptidase T PDI is involved in controlled proteolysis of proteins, namely apoptotic processes. Inside the endoplasmic reticulum PDI interacts under certain conditions with ubiquitin which possesses an ubiquitin-like domain and an ubiquitin-associated domain. This interaction is functionally connected with gaining tolerance to ischemic stress andapoptosis (Ko H. S. et al., 2002, J. Biol. Chem. 277: 35386-92).

The relationship of the two enzymes with regard to apoptosis makes an observation of their up and downregulation suitable as a characteristic marker for cancerous tissue. On the other hand it may be advantageous to analyze the abundance of only one of the proteins, especially ubiquitin isopeptidase T as a diagnostic marker. A great advantage is that ubiquitin isopeptidase T in cancer tissue is remarkably downregulated and displays only about one fifth to about one sixth of the abundance of healthy controls. The observed reduced abundance of ubiquitin-isopeptidase T in cancerous tissue is more strongly marked than in the case of fatty acid-binding protein of mammals (M-FABP) which is a recognized anti-oncogen.

A possible link between the influencing of T-cell activity by annexins through the MHC (main histocompatibility complex) antigen presentation via ubiquitin-isopeptidase T and through a changed activity of the systemic immune system through the absence of the immunoglobulin domain-containing SAP in prostate tissue could be very important for the survival of tumour cells in the presence of the immune system

The invention also covers the use of mitochondrial enoyl-coenzyme A-hydratase as diagnostic marker for cancer and/or as a therapeutic target molecule. This protein can also be used in combination with the fatty acid-binding protein 3 (FABP-3) and/or the epidermal fatty acid-binding protein (E-FABP) and/or annexin A3. Particular preference is given to an upregulation of mitochondrial enoyl-coenzyme A-hydratase and/or epidermal fatty acid-binding protein (E-FABP) and/or a downregulation of the fatty acid-binding protein 3 (FABP-3) and/or annexin A3 is performed in comparison with controls, because according to the invention such an upregulation/downregulation of these proteins is revealed as a characteristic feature for cancerous tissue

The inventors have been able to show that mitochondrial enoyl-coenzyme A-hydratase in cancerous tissue has its abundance increased by on average approximately 2.8 to 4 times. This enzyme has already been described in conjunction with β-oxidation of fatty acids and this mainly takes place in the mitochondria. Enoyl-coenzyme A-hydratase participates in the non-oxidative metabolism. It has long been known that even in the presence of an oxygen excess cancer cells have an increased, non-oxidative metabolism and that both fatty acid oxidation and de Novo synthesis increases in cancer patients. Cancer is brought into context with numerous changes in the fatty acid metabolism. Recently fatty acid synthase, the enzyme which is responsible for de Novo fatty acid synthesis has been proposed as a therapeutic pharmaceutical target. The present results show that enoyl-coenzyme A-hydratase is a similar suitable target. This link with the fatty acid metabolism represents a functional connection between enoyl-coenzyme A-hydratase and FABP-3 and E-FABP. The abundance of these fatty acid-binding proteins in cancerous tissue is also characteristically modified. FABP-3 is roughly downregulated 2.5 times and E-FABP upregulated roughly 2.3 times. Apart from the link with fatty acids, a role in connection with cell cycle control has been described for FABP-3 (Seidita G. et al 2000, Carcinogenesis 21: 2203-10). E-FABP has already been described in connection with different types of cancer and has been detected in the urine of cancer patients (Brouard M. C. et al. 2002, Melanoma-Research 12: 627-31). The increased abundance of this protein observed by the inventors makes it particularly suitable as a diagnostic marker for cancer when combined with the other markers mitochondrial enoyl-coenzyme A-hydratase and/or FABP-3 and/or annexin A3, because there is a functional link here between these different proteins. Thus, the abundance of one or in particularly advantageous manner two or three of these proteins can be observed in comparison with controls, so that through the characteristic up/downregulation of these proteins conclusions can be drawn regarding the existence of cancerous tissue. The link with the fatty acid metabolism also applies to further proteins described here, as will be indicated hereinafter.

The invention also covers the use of the protein serum-amyloid P-component (SAP) as a diagnostic marker or therapeutic reagent for cancer. The inventors were able to show that this protein in cancer tissue on average reveals an approximately 2.7 to 5.1 times reduction in its incidence. SAP is mainly found on stromal cells of benign prostate tissue so that its relatively lower incidence in cancerous tissue could be explained by the relatively smaller quantity of stromal cells in cancerous tissue. Therefore the investigation of the downregulation of SAP compared with controls is particularly suitable as a characteristic feature for cancerous tissue. SAP is a lectin-like acute phase protein (results from mice) of the pentraxine family and is linked with several amyloid clinical pictures. Amyloid deposits are sometimes observed in the male urological system, but there is scarcely an understanding of the biology thereof. Correctly folded native SAP, over and beyond on amyloid fibrils is also bound to polysaccharides, including microbial polysaccharides and matrix components, via acid carbohydrate determinants, phosphoethanol amine and phosphocholine. SAP is a constituent of simple membranes and possibly brings about their interactions with laminins and phospholipids. It participates in target recognition by phagocytes of evolutionary or systemic immune system, e.g. polymorphonuclear leucocytes and is bound to phospholipids on apoptotic cells and brings about their phagocytosis by macrophages. It has long been known that the SAP level in malignant human serum is increased and at least in the serum of some cancer patients IL-6 appears to be responsible for this. In summarizing, it can be assumed that SAP participates in the modulation of the interaction of non-cancerous cells with their environment and possibly immune monitoring: a function which is probably disturbed in many cancer cell. Pentraxines can be induced by cytokines and their concentration in the blood rises dramatically during infections or trauma, so that they play a part in immune defence. The present observation suggest a link between annexin A3 ubiquitin-isopeptidase T an the serum-amyloid P component in immune monitoring of the prostate leading to a modified regulation of immune monitoring by exosomes.

The invention also covers the use of the protein 14-3-3 protein tau as a diagnostic marker for cancer. This protein is known to participate in apoptotic processes. This process has already been described in conjunction with cancer, but an anti-oncogen nature was established (He H. 1997, Gan-To-Kagaku-Ryoho 24: 1448-53). However, the inventors have surprisingly found an increased level (1.8 times) of 14-3-3 protein tau in cancerous tissue. Immunohistochemical staining reactions have revealed that protein 14-3-3 tau mainly occurs in healthy epithelial cells and in cancer cells of the prostate tissue. However, in the stroma protein 14-3-3 tau only occurs in lymphocytes (only lymphocytes are stained). Thus, according to the invention there is an investigation of the upregulation of the protein compared with controls as the characteristic feature for cancerous tissue.

The invention also covers the use of the protein nuclear chloride ion channel protein (CLIC-1) as a diagnostic marker for cancer, particularly prostate cancer. The inventors established an approximately 1.5 times increase in the abundance of this protein in cancerous tissue compared with controls. Therefore preferably there is an investigation of an upregulation of this protein compared with controls as a characteristic feature for cancerous tissue. This intracellular anion channel was already described in connection with cell division and apoptosis (Ashley R. H., 2003, Mol. Membr. Biol. 20: 1-11).

Furthermore the invention covers application of the protein HES1 as diagnostic marker for cancer. The inventors could demonstrate that the abundance of this protein is about 4 fold higher in cancer tissue compared to controls. The probability of a corresponding upregulation has a p-value<0.0001 in a t-test. Preferentially upregulation of this protein compared to controls is considered a characteristic marker for cancer diseases. This protein is a certain splicing variant (HES1/Kpn-la) with unknown functions. It contains a DJ1-Pfdl-domain; it is supposed to be located in mitochondria which could indicate a possible link with the function of enoyl-coenzyme A-hydratase. This protein is expressed in a number of human tissues. Its connection to cancer has been demonstrated for the first time by the inventors.

Furthermore the invention covers application of proteasome alpha 2 subunit as a diagnostic marker for cancer. Also for this protein the connection to cancer diseases has been demonstrated for the first time by the inventors. In cancer tissue the abundance was doubled compared to controls. A t-test for cancer-related changes of this protein were significant with p<0.009. Preferentially upregulation of this protein compared to controls is examined. Proteasomes are well known for their function in processing peptides for antigen presentation in the MHC class 1 system, which contributes to the activity of killer t cells.

Furthermore the invention covers application of the protein adenine-phosphoribosyltransferase as a diagnostic marker for cancer, especially prostate cancer. The connection of this protein to cancer has been discussed recently. For example downregulation of this protein in lymphocytes of breast cancer patients has been described. Furthermore overexpression of the protein in colorectal carcinoma has been observed. The inventors could demonstrate that the abundance of this protein is about 2 fold higher in prostate cancer tissue compared to controls. These results are significant in a t-test for differential expression with p<0.007. According to the invention upregulation of this protein compared to controls is considered a characteristic marker for cancer tissue.

Furthermore the invention covers application of the protein inorganic pyrophosphatase as a diagnostic marker for cancer, especially prostate cancer. Upregulation of this protein in lung cancer an colorectal cancer has been shown recently. The inventors could demonstrate that the abundance of this protein is 1.6 fold higher in prostate cancer tissue compared to normal tissue. These results are significant in a t-test for differential expression with p<0.005. Inorganic pyrophosphatase 1 catalyzes a reaction that releases inorganic phosphate. This relates to processes of calcification in which annexins, especially annexin A3 are involved, which participate in the Ca²⁺ flow. Particularly a functional relationship exists therefore between upregulation of annexin A3 and upregulation of inorganic pyrophosphatase 1.

The various proteins referred to herein as well as the proteins further mentioned in the following may be used for diagnostic purposes alone or in combination with other proteins.

Furthermore the invention covers application of at least one of the following proteins as diagnostic markers for cancer: ubiquitin-isopeptidase T, serum amyloid P component (SAP), fatty acid-binding protein 3 (FABP-3), galectin-1, heat shock protein 27 (HSP27), 14-3-3 protein beta, 14-3-3 protein zeta, nuclear chloride ion channel protein 1 (CLIC-1) 14-3-3 protein tau, heat shock protein 90 (HSP 90), protein-disulphide-isomerase (PDI), epidermal fatty acid-binding protein (E-FAPB), mitochondrial enoyl-coenzyme A hydratase, nucleophosmin annexin, especially annexin A3, transgelin, triosephosphate-isomerase, aldolase A HES 1, alpha 2-subunit of the proteaesome, adenine-phosphoribosyl-tansferase. Preferentially downregulation of at least one of the proteins isopeptidase T, serum-amyloid P-component (SAP), fatty acid-binding protein 3 (FABP-3), galectin-1, microseminoprotein beta, heat shock protein 27 (HSP27) or transgelin compared to controls is considered a characteristic marker for cancer disease. Furthermore preferentially a supplementary or alternative upregulation of at least one of the proteins 14-3-3 protein beta, 14-3-3 protein zeta, nuclear chloride ion channel protein 1 (CLIC-1), 14-3-3 protein tau, heat shock protein 90 (HSP 90), protein-disulphide-isomerase (PDI), epidermal fatty acid-binding protein (E-FAPB), mitochondrial enoyl-coenzyme A hydratase, nucleophosmin, annexin, especially annexin 3, triosephosphate-isomerase, aldolase A, HES 1, alpha 2-subunit of the proteaesome, adenine-phosphoribosyl-transferase and inorganic pyrophosphatase 1 compared to controls is considered a characteristic marker for cancer disease. In particularly preferred manner, in addition to one or more of these proteins there is an investigation of the downregulation of other annexins. It is particularly preferred to investigate at least two proteins.

A particular advantage is offered by the application of two of the following proteins as diagnostic markers: ubiquitin-isopeptidase, heat shock protein 27 (HSP27) heat shock protein 90 (HSP90), protein-disulphide-isomerase (PDI) mitochondrial enoyl-coenzyme A hydratase and/or nucleophosmin.

According to the invention it has been demonstrated, that the expression of a set of distinct proteins is characteristically down- or upregulated respectively. Details are shown in the following table 1 which summarizes the results of identification and quantification of proteins differentially expressed between benign and malignant tissue. The selection of proteins is based on a statistically significant differential expression analysis of the proteins in benign (benign fraction) or malignant tissue (cancer fraction). The accession number refers to the respective number in the NCBI database. The theoretical molecular weight (MW) was calculated from the sequences in the database. “Scores” means hits determined with MASCOT techniques. The details given about the PMF-score refer to a Mouse-score which is used by the MASCOT-server; generally a PMF-score over 65 represents significant identification. The last two columns summarize the quantification of the intensities of the protein spots that were found in benign and malignant tissue samples.

TABLE 1

| | | | Theor. | PMF | Benign | Cancer |
|---|---|---|---|---|---|---|
| No | AccNo | Description | MW | Score | fraction | fraction |
| 1 | gi\|1732411 | isopeptidase T [Homo sapiens] | 94104 | 115 | 83.6 | 16.4 |
| 2 | gi\|576259 | Chain A; Serum Amyloid P Component (Sap) | 23598 | 106 | 73.1 | 26.9 |
| 3 | gi\|494781 | Fatty Acid Binding Protein (Holo Form, Human Muscle) | 14775 | 87 | 71.6 | 28.4 |
| | | (M-Fabp) | | | | |
| 4 | gi\|4504981 | beta-galactosidase binding lectin precursor; Lectin; galactose- | 15769 | 177 | 66.2 | 33.8 |
| | | binding; soluble; 1; galectin [Homo sapiens] | | | | |
| 5 | gi\|225159 | microseminoprotein beta | 12238 | 92 | 63.9 | 36.1 |
| 6 | | n.i. | | | 60.6 | 39.4 |
| 7 | gi\|662841 | heat shock protein 27 [Homo sapiens] | 22667 | 182 | 60.2 | 39.8 |
| 8 | gi\|4507949 | tyrosine 3-monooxygenase/tryptophan 5-monooxygenase | 27946 | 160 | 41.2 | 58.8 |
| | | activation protein, beta polypeptide; 14-3-3 pr | | | | |
| 9 | gi\|4507953 | tyrosine 3/tryptophan 5-monooxygenase activation protein, | 27810 | 160 | 41.1 | 58.9 |
| | | zeta polypeptide; protein kinase C inhib | | | | |
| 10 | gi\|2073569 | nuclear chloride ion channel protein [Homo sapiens] | 27249 | 139 | 40.1 | 59.9 |
| 11 | | n.i. | | | 39.5 | 60.5 |
| 12 | | n.i. (Annexin A3) | 36524 | 160 | 37.4 | 62.6 |
| 13 | gi\|5803227 | tyrosine 3/tryptophan 5-monooxygenase activation protein, | 28032 | 130 | 35.6 | 64.4 |
| | | theta polypeptide, 14-3-3 protein tau | | | | |
| 14 | gi\|13129150 | heat shock 90 kDa protein 1, alpha, heat shock 90 kD protein | 85006 | 147 | 32.6 | 67.4 |
| | | 1, alpha [Homo sapiens] | | | | |
| | gi\|20149594 | heat shack 90 kDa protein 1, beta, heat shock 90 kD protein | 83554 | 164 | | |
| | | 1, beta, Heat-shock 90 kD protein-1, beta | | | | |
| 15 | gi\|20070125 | prolyl 4-hydroxylase, beta subunit, v-erb-a avian erythroblastic | 57480 | 235 | 31.2 | 68.8 |
| | | Leukaemia viral oncogene homolog 2 | | | | |
| 16 | gi\|4557581 | (NM_001444) fatty acid binding protein 5 (psoriasis- | 15497 | 94 | 27.9 | 72.1 |
| | | associated); E-FABP [Homo sapiens] | | | | |
| 17 | gi\|12707570 | mitochondrial short-chain enoyl-coenzyme A hydratase 1 | 31807 | 101 | 26.2 | 73.8 |
| | | precursor [Homo sapiens] | | | | |
| 18 | gi\|16307090 | Similar to nucleophosmin (nucleolar phosphoprotein B23, | 29617 | 77 | 21.9 | 78.1 |
| | | numatrin) [Homo sapiens] | | | | |
| 19 | gi\|7768772 | HES1 protein, homolog to E. coli and zebrafish ESI protein, | 29215 | 95 | <20 | >80 |
| | | anti-sigma cross-reacting protein homolog I alpha | | | | |
| | | precursor, KNP-Ia, GT335, similar to E. coli SCRP27A | | | | |
| | | and to zebrafish ESI [Homo sapiens] | | | | |
| 20 | gi\|4506181 | proteasome alpha 2 subunit; proteasome subunit HC3; | 26236 | 105 | 32.6 | 67.4 |
| | | proteasome component C3; macropain subunit C3; multi- | | | | |
| | | catalytic endopeptidase complex subunit C3 [Homo sapiens] | | | | |
| 21 | gi\|4502171 | adenine phophoribosyltransferase; AMP pyrophosphorylase; | 20127 | 134 | 33 | 67 |
| | | AMP diphosphorylase; transphosphoribosidase | | | | |
| 22 | gi\|11056044 | inorganic pyrophosphatase [Homo sapiens] | | | 38.6 | 61.4 |

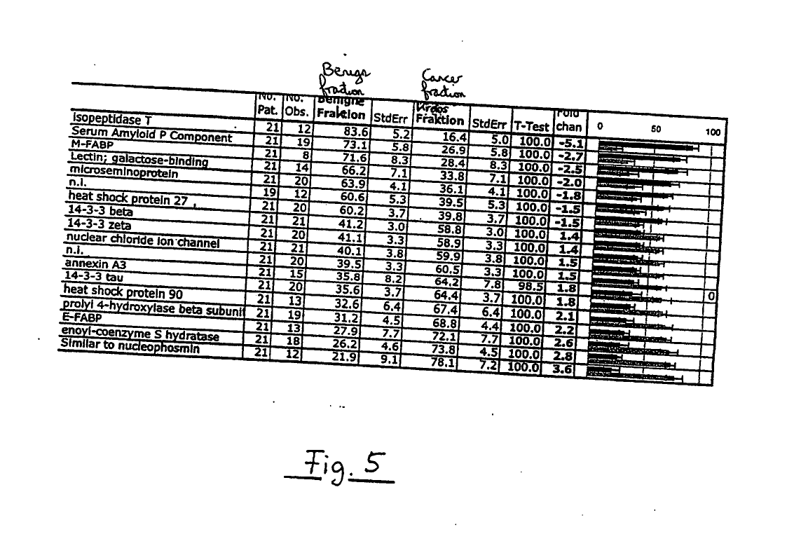

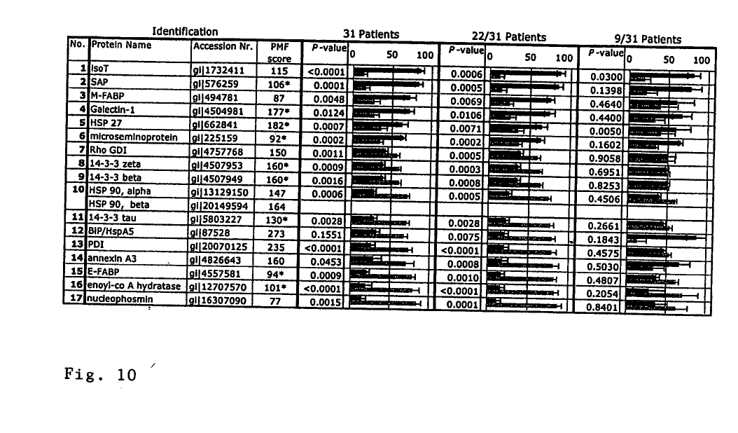

In this context we refer to FIG. 5 and FIG. 10 that show in tabular form the results of protein spots with significant average differential expression for 21 patients and 31 patients together with statistical data.

A survey of English synonyms for the different proteins is listed below. The numbers put in front correspond to the numbering of table 1.
1. gi|1732411: Ubiquitin-isopeptidase T; Isopeptidase T (sioT); ubiquitin specific protease 5; ubiquitin carboxyl-terminal hydrolase 5; ubiquitin thiolesterase 5; ubiquitin-specific processing protease 5; deubiquitinating enzyme 5; de-ubiquitinase.
2. gi|576259: Serum-amyloid P-component; Chain A; Serum amyloid P Component (SAP).
3. gi|494781: Fatty acid-binding Protein 3 (FABP-3); Mammaryderived growth inhibitor (MDGI); fatty acid binding protein 3 (FABP-3); Heart-Type fatty acid binding protein (H-FABP); Muscle type fatty acid binding protein (M-FABP).
4. gi|4504981: Galectin; galectin-1; kDa beta-galactoside-binding lectin; beta galactoside soluble lectin; beta-galactoside-binding lectin 1-14-1; galaptin; soluble galactoside binding lectin; S-Lac lectin 1.
5. gi|225159: Microsamine protein beta; beta-microseralnoprotein; microseminoprotein beta; Immunoglobulin binding factor (IGBF); PN44; Prostate sacrated seminal plasma protein; Prostate secretory protein of 94 amino acids (PSP-94); Seminal plasma beta-inhibin; seminal plasma protein.
6. not identified.
7. gi|662841: Heat shock protein 27 (HSP27); heat shock protein 27; 27 kDa heat shock protein 1 (HSP-27); Stress-responsive protein 27 (SRP237); Estrogen-regulated 24 kDa Protein; 28 kDa heat shock protein.
8. gi|4507949: 14-3-3 Protein beta; 14-3-3 protein beta (14-3-3 beta); 14-3-3 protein alpha (14-3-3 alpha); Protein kinase C inhibitor protein-1; PKC inhibitor protein-1 (KCIP-1: also 14-3-3 zeta); RNH-1.
9. gi|4507953: 14-3-3 Protein zeta; 14-3-3 zeta; 14-3-3 delta; KCIP-1 (also 14-3-3 beta); YWHAZ; mitochondrial import stimulation factor S1 (MSF S1); Factor activating exoenzyme S; tryptophan monooxygenase activation protein zeta; tyrosine monooxygenase activation protein zeta.
10. gi|2073569: Nuclear chloride ion channel protein; chloride intracellular channel 1 (CLIC-1); nuclear chloride ion channel protein (p64CLCP); nuclear chloride channel; chloride channel ABP; nuclear chloride ion channel 27 (NCC27); RNCC protein; nuclear chloride ion channel 27 (NCC27).
11. not identified.
12. (Annexin A3, see 23).
13. gi|5803227: 14-3-3 Protein tau; 14-3-3 theta; S15076 protein kinase regulator 14-3-3; HS1; tryptophan 5-monooxygenase activation protein; tyrosine 3-monooxygenase activation protein.
14. gi|13129150: Heat shock protein 90 (HSP90); heat shock protein 90 (HSP0-90); heat shock protein HSP 90-alpha; heat shock protein 90-alpha; 90 kDa heat shock protein; heat shock protein 86 (HSP86); Hspca; heat shock 90 kDa protein 1; heat shock protein 1; tumor specific transplantation 86 kDa antigen (TSTA).
15. gi|20070125: Protein disulphide isomerase (PDI); protein disulphide isomerase (PDI); protyl-4-hydroxylase beta; protein disulphide oxidoreductase; thyroid hormone binding protein p55; glutathlone insulin transhydrogenase.
16. gi|4557581: Epidermal fatty acid-binding protein (E-FABP); fatty acid binding protein 5 (FABP-5); epidermal fatty acid-binding protein (E-FABP); Psoriasis-associated fatty acid-binding protein (PA-FABP); cutaneous fatty acid-binding protein (C-FABP); keratinocyte acid-binding protein (KLBP); DA11.
17. gi|2707570: Mitochondrial enoyl-coenzyme-A-hydratase; Mitochondrial enoyl coenzyme A hydratase; Mitochondrial enoyl-CoA hydratase; short-chain enoyl-CoA hydratase, mitochondrial; short-chain enoylcoenzyme A hydratase (SCEH).
18. gi|6307090: Nucleophosmin; nucleophosmin; nucleolar phosphoprotein B23; nucleolar protein NO38; numatrin; NPM(1).
19. gi|7768772: HES1 protein, homolog to E. coli and Zebra fish ES1 protein; anti-sigma cross-reacting protein homolog 1 alpha precursor, KNP-la/Kpn-1 alpha, GT335 (similar to E. coli SCRP27A and to Zebra fish ES1 [(Homo sapiens].
20. gi|4506181: Proteasome alpha 2-subunit; proteasome subunit HC3, proteasome component C3; macropain subunit C3; multicatalytic endopeptidase complex subunit C3 [Homo sapiens].
21. gi|4502171: Adenine-phosphoribosyltransferase; AMP pyrophosphorylase; AMP diphosphorylase; transphosphoribosidase.
22. gi|11056044: Inorganic pyrophosphatase; cylosolic inorganic pyrophosphatase; inorganic pyrophosphatase 1; pyrophosphate phospho-hydrolase [Homo sapiens].

Moreover four more proteins were identified which are up- or downregulated in cancer tissue as compared to controls in certain patient collectives (cluster analysis). These are the proteins annexin A3, transgelin, triosephosphate isomerase and aldolase A. In cancer tissue annexin A3 is upregulated about 5-fold and transgelin is downregulated about 5-fold. Triosephosphate isomerase and aldolase A are upregulated about 20% and 10% respectively in cancer tissue.

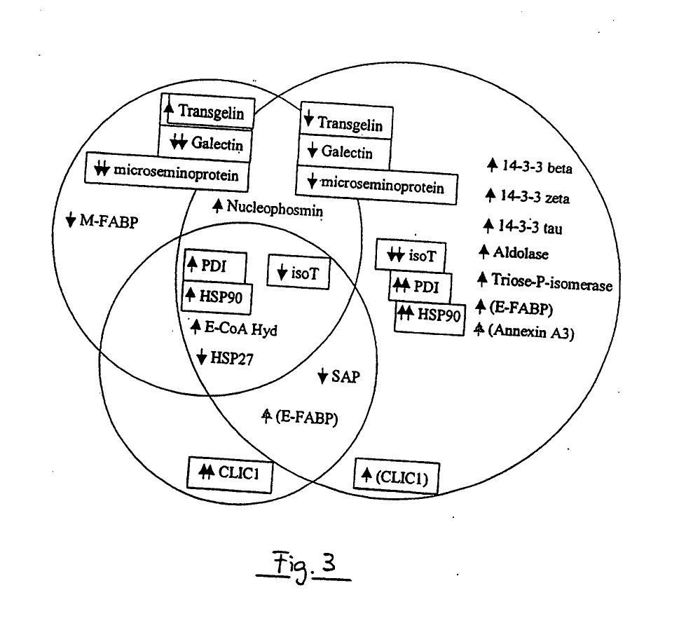

In this respect we refer to FIG. 3 that shows a graphical representation of the results of the cluster analysis. The figure illustrates up- and downregulation of different proteins in cancer tissue of certain patient groups (or clusters respectively) each represented by a circle, in comparison to healthy tissue.

The English synonyms for annexin and transgelin are the following:
23. gi|4826643: Annexin A3; Annexin III; Lipocortin III; anticoagulant protein III; Placental aticoagulant protein III (PAP III); 35 alpha calcimedin.
24. gi/4507359: Transgelin; SM22-alpha smooth muscle protein, 22 Da actin-binding protein, smooth muscle 22 protein, actin-associated protein p27, 25 kDa F-actin-binding protein.

In addition further proteins were identified which showed differing levels of abundance in certain patient groups (down or upregulation) in cancer tissue compared to controls. These proteins were ATP synthase, biliverdin reductase B, glucose-regulated protein, prolyl-4-hydrolase beta and dnak-like molecular chaperon. ATP synthase is downregulated, the other proteins are upregulated.

Interestingly many of the proteins we identified are related to lipid metabolism. Direct binding of annexin A3 and SAP to lipids has been reported. Both proteins are involved in phagocytosis. FABP-3 and E-FABP are fatty acid binding proteins. Mitochondrial enoyl-coenzyme A hydratase is involved in β-oxidation of fatty acids. Phospholipases that react with phospholipids induce protein kinase C which stimulates the activity of HSP 27. HSP 90 is also involved in phosopholipid metabolism because inhibition of HSP 90 results in changes of the phospholipid metabolism (Chung Y. et al., 2003, J. Natl. Cancer Inst. 95: 1624-33). Furthermore it is supposed that PDI is also linked to the metabolism of lipids because it acts as a multifunctional protein and—among others—takes part in triglyceride transfer (Horiuchi R. and Yamauchi K., 1994, Nippon-Rinsho 52: 890-5). Moreover 14-3-3 proteins inhibit proteinkinase C and contain conserved sequences which look like the pseudo-substrate domain of proteinkinase C and the C-terminus of annexins. This indicates a functional relationship among those different proteins.

According to the invention the diagnostic markers can be used to identify different types of tumours and cancerous diseases. In a preferred embodiment of the invention the cancer disease that shall be diagnosed is prostate cancer, especially a prostate carcinoma. As mentioned earlier, carcinomas of the prostate gland are the most common malignant tumours in men. Only when detected at an early stage prostate cancer can be successfully treated by prophylactic surgical removal of the prostate gland. If the disease progresses and is no longer limited to one organ prophylactic removal of the prostate gland is not sufficient. For prostate tumours that cannot be removed by surgery, inhibition of male sex hormones can be taken into account. This inhibition, preferably with surgical or pharmacological castration may sometimes inhibit proliferation and metastasis of the tumour and allows tumour control for a certain time. But most prostate tumours become resistant to this endocrinological therapy after a while. Other therapeutic means e.g. applications of cytotoxic agents, gene therapy or immunotherapy are still under clinical investigation and have not yet been successful. Therefore it is necessary to detect a tumour of the prostate gland as early as possible in order to be able to remove it successfully by surgery. According to the invention the described marker proteins offer great advantages for the early detection of prostate cancer.

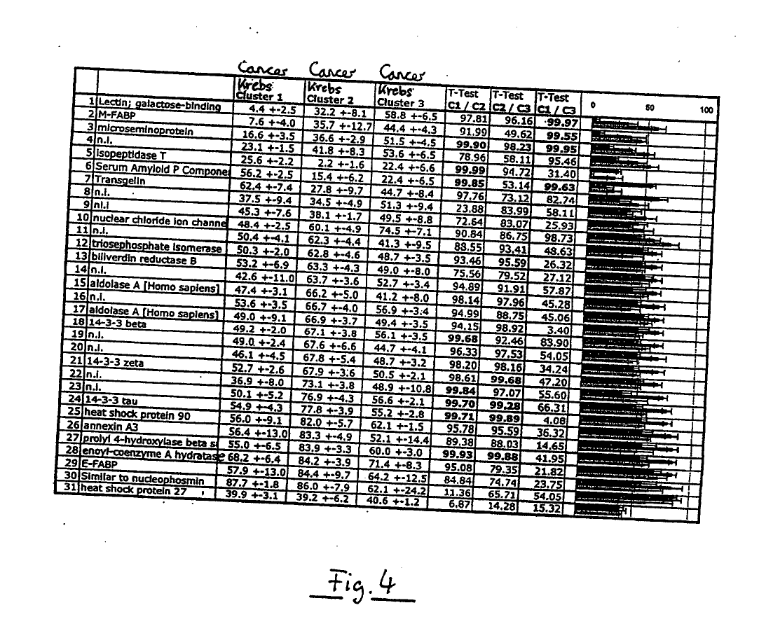

In a preferred embodiment of the inventive method a certain subtype of cancer, especially a subtype of prostate cancer can be diagnose by quantification of preferably several of the mentioned proteins. The inventors could demonstrate that by means of a so-called cluster analysis distinct protein patterns reflect a characteristic up- or downregulation of different proteins which correlate to distinct patient collectives. The patients belonging to a certain collective all show the same distinct subtype of cancer, especially prostate cancer. According to the invention it is intended that patients will be characterized according to certain subtypes of cancer in relation to the protein pattern determined by application of the inventional method in order to treat this subtype of cancer selectively. Preferably defined combinations of proteins should be analyzed. In this respect we refer to FIG. 3 that shows a graphical representation of characteristic protein patterns corresponding to the different patient collectives. The table in FIG. 4 illustrates a summary of protein patterns which represent the different patient collectives and the subtypes of prostate cancer respectively.

In order to diagnose the different subtypes of prostate cancer preferably the abundance of a combination of different proteins should be determined. Therefore at least one of the following should be determined as a common cancer marker: upregulation of nucleophosmin, protein disulphide isomerase, heat shock protein 90, mitochondrial coenzyme A hydratase; downregulation of heat shock protein 27 and/or ubiquitin isopeptidase T. These should be analyzed together with at least one of the following proteins for the three subtypes of prostate cancer.
Subtype a: Upregulation of transgelin; substantial downregulation of galectin and microseminoprotein beta; downregulation of fatty acid binding protein 3; no or minor changes of epidermal fatty acid-binding protein, no or minor changes of nuclear chloride ion channel protein, 14-3-3 protein beta, zeta and tau, aldolase A, serum amyloid P component, triosephosphate isomerase and/or annexin A3.
Subtype b: Substantial upregulation of protein disulphide isomerase, heat shock protein 90; substantial downregulation of ubiquitin isopeptidase T; upregulation of 14-3-3 protein beta, zeta and tau, aldolase A, triosephosphate isomerase, annexin A3; downregulation of transgelin, galectin microseminoprotein beta, serum amyloid P component; no or minor changes of fatty acid binding protein 3 and/or nuclear chloride ion channel protein.
Subtype c: Substantial upregulation of nuclear chloride ion channel protein; downregulation of serum amyloid P component; no or minor changes of fatty acid binding protein 3, 14-3-3 protein beta, zeta and tau, aldolase A, triosephosphate isomerase, annexin A3, epidermal fatty acid-binding protein; microseminoprotein beta, galectin, transgelin.

According to the invention, for diagnosing the different subtypes of prostate cancer, it is possible to analyze at least one general cancer marker combined with at least annexin A3 as a further protein.

In another, particularly preferred embodiment through the exclusive investigation of annexin A3 and/or mitochondrial enoyl-coenzyme A-hydratase it is possible to diagnose a specific prostate cancer subtype occurring in specific patient groups.

In the case of the inventive use of the described proteins as diagnostic markers use can be made of various methods in order to analyze the incidence or abundance of the protein in cancerous tissue (or in the tissue under investigation) compared with control tissue. It is particularly advantageous if the proteins of the sample to be investigated and the control sample are separated gel electrophoretically, e.g. on a conventional polyacrylamide gel. Then the abundance of the given proteins in the sample and control are compared. Due to the necessary decomposition two-dimensional gels are particularly preferred. However, it is also possible to carry out a prepurification prior to gel electrophoretic separation so that an adequate separation and analyzability can be obtained e.g. with a one-dimensional polyacrylamide gel electrophoresis. Other protein separation methods can also be use with advantage, e.g. conventional chromatographic methods, particularly column chromatographic methods. It is particularly advantageous if the sample to be investigated and the control sample are marked or labelled in different ways, e.g. using different isotopes. This facilitates a comparison of the sample to be investigated and the control with respect to the abundance of the given proteins. In another preferred embodiment the proteins to be analyzed are examine mass spectrometrically in order to permit a precise identification of the proteins. Thus, e.g. the surface enhanced laser desorption ionization method (SELDI method) can be used in tissues or body fluid preparations. However, it is also advantageously possible to use in vivo image-giving methods, particularly positron emission tomography (PET).

The proteins to be investigated are also qualitatively and quantitatively characterized with the aid of molecules which are directed counter to the proteins under investigation and which are used as diagnostic markers. In particularly advantageous manner the molecules are antibodies, particularly polyclonal and/or monoclonal antibodies. However, the invention also covers all known affinity reagents in this connection.

For qualitative and in particular quantitative identification conventional immunoassays can be used such as e.g. enzyme-linked immunoabsorbent assays (ELISA). It is also possible to use immunohistochemical methods and/or protein chips, e.g. also the SELDI method. Body fluids or tumour tissue can e.g. be investigated for identification purposes. Antibodies are particularly suitable for identifying annexin A3 14-3-3 protein beta, tau and zeta and/or SAP. For example, pan anti 14-3-3 beta/zeta monoclonal antibody (Stressgen catalogue number KAM-CC012C) stains epithelial an cancer cells, as well as certain lymphocytes in the stroma. The stroma, but not epithelial or cancer cells are stained by monoclonal antibodies against the protein serum-amyloid P component (SAP) (Stressgen catalogue number HYB 281-05, working dilution 1:10).

In a further preferred embodiment, qualitative and quantitative measurements of diagnostic marker proteins are carried out with the help of oligonucleotides, e.g. during a common polymerase chain-reaction (PCR). PCR belongs to the methodological repertoire of molecular genetics that selectively amplifies determined DNA-sequences. The method delivers qualitative and quantitative detection of the test proteins on the DNA- and RNA-level respectively. Using suitable oligonucleotides hybridization assays, e.g. common Northern- or Southern blots are possible; they also give qualitative and quantitative information about the proteins on the DNA- or RNA-level. Methods for detection with oligonucleotides can easily be automated, that is one of their advantages. On the other hand they only deliver information about the abundance of a certain DNA- or RNA-sequence and not about the real abundance of the corresponding proteins. In this case it is necessary to make sure that the characteristic up- and downregulation of the diagnostic marker proteins is achieved on the mRNA-level or if the regulation takes place on a level following transcription.

The characteristic changes of the abundance of different marker proteins as determined according to the invention also affects the activities of the respective proteins, e.g. their enzymatic activity. Therefore it should be an advantage to determine the activity of the proteins alternatively or in parallel to their abundance as compared to controls. This also is understood by the term up- or downregulation of the various proteins. A respective determination can be done by common enzymatic tests for the respective enzymes which are clear to the experts. Furthermore binding assays or comparable tests can be performed with fatty acid-binding proteins, in order to get information about their activity and their up- or downregulation respectively. The same goes for the other proteins, e.g. channel activities of nuclear chloride ion channel protein (CLIC-1) can be measured. This can be used for the application of the various proteins as diagnostic markers or in the diagnostic kit described according to the invention in the following. Moreover measuring the activities of the respective proteins can be used to test the effect of drugs for cancer treatment according to the invention, as described in the following:

In a further preferred embodiment of application according to the invention for examination of at least one protein, exosomes e.g. from patient material are isolated and analyzed with regard to the protein(s) of interest. Especially in the protein pattern corresponding to at least one protein inside the exosomes will be tested to determine the diagnostically relevant up- and/or downregulation of one or more proteins. A suitable method for preparation of-exosomes from patient material may be done with standard methods, which are known to the experts.

Furthermore the invention covers a diagnostic kit which contains at least one compound for the determination of activity and/or expression of at least one of the proteins reported as diagnostic markers according to the above mentioned description. This diagnostic kit is preferentially used to determine activity and/or expression of at least one of the following proteins: isopeptidase T, serum amyloid P component (SAP), nuclear chloride ion channel protein channel 1 (CLIC-1), mitochondrial enoyl-coenzyme A hydratase and/or annexin A3. Above all such a diagnostic kit serves for the determination of the respective abundance of at least one of these proteins which is characteristically up- or downregulated compared to controls. First and foremost abundance reflects the expression of the protein. The diagnostic kit developed according to the invention is preferentially suitable for detection or screening of cancerous diseases, especially prostate cancer; it offers special benefits for the early diagnosis of these diseases. For example such a diagnostic kit allows for the discrimination of benign or healthy tissue and malignant tissue, e.g. benign tissue in prostate hyperplasia or prostate cancer. Preferably such a kit contains one or several antibodies or one or several oligonucleotides or pairs of oligonucleotides respectively which interact with one or more of the described proteins or the related nucleic acids. With the help of these compounds qualitative and especially quantitative information about the proteins compared to controls may be gained.

The samples to be tested and the controls are taken from the same patient. For example tissue samples or samples of body fluids like blood, lymph or urine are taken and prepared by methods familiar to the experts. Preferentially, potentially malignant tissue, i.e. the sample that shall be tested, and control tissue, i.e. benign tissue are taken from the same patient and compared directly. On the other hand it is also possible to compare the abundance of the respective proteins to other standards which have been determined statistically from a great number of independent control samples. In case of prostate cancer it will be an advantage to take benign and potentially malignant prostate tissue from a patient whose prostate had been removed by surgery. Benign tissue from prostate hyperplasia may serve as a control.

Furthermore the invention covers a method for diagnosing cancerous diseases by analyzing the abundance of at least one of the described proteins. The results of an analysis of their up- and downregulation in cancerous tissue according to the invention deliver information about existing cancer tissue. With regard to other characteristics of the inventive method we refer to the above mentioned description.

The invention also covers the use of at least one active substance which interacts with the protein annexin A3 and in particular influences and preferably inhibits the activity and/or abundance of annexin, particularly annexin A3, in order to produce a medicament for the treatment of prostate cancer, preferably specific prostate cancer patient groups. According to the invention it is preferable for the active substance to interact directly with the protein annexin A3 and in this way to influence, preferably inhibit its activity and/or abundance. In another embodiment of the invention, it can be advantageous for the active substance to interact indirectly with the protein annexin A3, in that the active substance is e.g. directed against activators, inhibitors, regulators and/or biological precursors of annexin A3.

In a special preferred embodiment of this application the active substance is at least one derivative of the benzodiazepine-type (Hofmann et al., 1998, J. Biol. Chem. 273 (5): 2885-94). Especially preferred are BDA250 (1,3-dihydro-1-methyl-5-phenyl-2H-1,4-benzodiazepin-2-one), BDA452 (3-(R,S)-(L-tryptophanyl)-1,3-dihydro-1-methyl-5-phenyl-2H-1,4-benzodiazepin-2-one) and/or BDA753 (3-(R,S)-all-L-(NH-Trp-Gly-Tyr-Ala-H)-1,3-dihydro-1-methyl-5-phenyl-2H-1,4-benzodiazepin-2-one). Furthermore the use/application of diazepam(7-chloro-1,3-dihydro-1-methyl-5-phenyl-2H-1,4-benzodiazepine-2-one) is preferred. Other molecules derived from these substances may preferably be used according to the invention. Especially those molecules are concerned that block the activity of annexin A3.

In particularly preferred manner an annexin A3-specific antibody is suitable as the active substance, particular preference being given to therapeutic antibodies. These are preferably blocking antibodies and/or radioactively labelled and/or toxin-labelled antibodies. The radioactively labelled antibodies can e.g. carry ¹³¹I. Such antibodies advantageously make it possible to carry out a radioimmunotherapy, as is known to the expert. However, any other reagent known to the expert is also suitable as an active substance.

In particularly preferred manner such active substances can be used for influencing the activity and/or abundance of annexin A3 in exosomes.

Active substances that influence activity and/or expression of annexin A3, especially those that display an inhibiting effect, can be advantageous for the production of a medicament for therapy of osteoarthritic degradation and/or atherosclerotic lesions.

Furthermore the invention covers the application of at least one active substance that influences activity and/or expression of isopeptidase T and/or activity and/or expression of protein-disulphide-isomerase (PDI) for the development of a medicament for cancer treatment. As described earlier, the abundance of these proteins in cancerous tissue has characteristically changed. Particularly the abundance of ubiquitin-isopeptidase T is significantly decreased and the abundance of protein-disulphide-isomerase (PDI) is increased. Altering expression or activity of these proteins to the normal level as it is shown in control tissue represents a possible way for curing cancer diseases. Thus, the use of an active substance is claimed which increases the activity and/or abundance of ubiquitin-isopeptidase T. In addition, the use of an active substance is claimed which inhibits the activity and/or abundance of PDI. Through such active substances the activity of said proteins is regulated to the normal level, so that a cancerous disease can be effectively treated.

The invention also covers the use of at least one active substance influencing the activity and/or abundance of mitochondrial enoyl-coenzyme A-hydratase for producing a medicament for the treatment of cancer. Preferably this can be carried out in combination with an influencing of the fatty acid-binding protein 3 (FABP-3) and/or the epidermal fatty acid-binding protein (E-FAPB). Preferably use is made of an inhibiting active substance for the activity and/or abundance of mitochondrial enoyl-coenzyme A-hydratase and/or E-FABP, respectively an increasing active substance for the activity and/or abundance of FABP-3.

The invention also covers the use of at least one active substance influencing and in particular increasing the activity abundance and/or localization of the serum amyloid P component (SAP) for producing a medicament for the treatment of cancer. It has been shown that the location of SAP in cancerous diseases can be modified. It is therefore inventively preferred for the localization of SAP to be influenced by the use of at least one active substance. The active substance can e.g. be a fusion protein comprising a cancer cell-binding domain of annexin A3 and an immunoreaction-influencing domain of SAP. SAP is e.g. located in prostate tissue on stromal cells, but not on healthy epithelial cells or transformed cancer cells. Annexin A3 is abundant in cancer tissue. It is also known that annexins appear on the surface of cells. As SAP as a protein component participates in the immune system and cancer cells are not eliminated from the immune system, an immune reaction-influencing domain of SAP on the surface of cancer cells could give rise to a modified immune reaction with respect to cancer cells.

Furthermore the invention covers the application of at least one active substance that influences—preferably inhibits—activity and/or expression of 14-3-3 protein tau for the development of a medicament for cancer treatment.

Furthermore the invention covers the application of at least one active substance that influences—preferably inhibits—activity and/or expression of nuclear chloride ion channel protein 1 (CLIC-1) for the development of a medicament for cancer treatment, especially prostate cancer.

Furthermore the invention covers the application of at least one active substance that influences—preferably inhibits—activity and/or expression of HES 1 for the development of a medicament for cancer treatment.

Furthermore the invention covers the application of at least one active substance that influences—preferably inhibits—activity and/or expression of alpha 2-subunit of the proteasome for the development of a medicament for cancer treatment, especially prostate cancer.

Furthermore the invention covers the application of at least one active substance that influences—preferably inhibits—activity and/or expression of adenine-phosphoribosyl-transferase for the development of a medicament for prostate cancer treatment.

Furthermore the invention covers the application of at least one active substance that influences—preferably inhibits—activity and/or expression of inorganic pyrophosphatase, particularly in exosomes, for the development of a medicament for prostate cancer treatment.

Furthermore the invention covers the application of at least one active substance that influences—preferably stimulates—activity and/or expression of at least one of the following proteins for the development of a medicament for prostate cancer treatment: ubiquitin-isopeptidase T, serum-amyloid P-component (SAP), fatty acid-binding protein 3 (FABP-3), galectin-1, microseminoprotein beta, heat shock protein 27 (HP27) and transgelin. Furthermore the invention covers the application of at least one active substance that influences—preferably inhibits—activity and/or expression of at least one of the following proteins for the development of a medicament for cancer treatment: 14-3-3 protein beta, 14-3-3 protein zeta, nuclear chloride ion channel protein 1 (CLIC-1), 14-3-3 protein tau, heat shock protein 90 (HSP 90), protein-disulphide-isomerase (PDI), epidermal fatty acid-binding protein (E-FABP), coenzyme A hydratase, nucleophosmin, annexin, especially annexin A3, triosephosphate-isomerase, aldolase A, alpha-2-subunit of the proteaesome, adenine-phosphoribosyl-transferase and inorganic pyrophosphatase. Special preference is given to the combination of two or more active substances against at least two different proteins. Moreover it is preferred that the active substance will be used together with one or more than one of these active substances which increases the activity and/or abundance of annexin A1, A2, A4, A7 and/or A10, particularly in exosomes.

According to the invention for each of these proteins, it was demonstrated that they are characteristically up- or downregulated in cancerous tissue compared to controls. Therefore the reverse down- or upregulation of these proteins by the respective active substances is claimed according to the invention in order to achieve activities, especially enzymatic activities of healthy tissues, or to inhibit and/or kill cancer cells. This has made it possible to successfully treat various cancerous diseases. It is particularly preferred to produce medicaments for the treatment of prostate cancer, preferably specific prostate cancer subtypes.

The active substances used according to the invention may be peptides, proteins, small molecular compounds or polynucleotides. Well known active substances with a well known mechanism of influencing the activity and/or expression of the different proteins are concerned as well as new active substances. These active substances can address the proteins described directly. On the other hand it can be advantageous if these active substances address regulators, especially activators or inhibitors and/or biological precursors of these different proteins. Depending on whether a certain active substance exerts inhibition or stimulation of the activity and/or expression of the respective protein, they can be agonists or antagonists. Further examples for antagonists are deficient or dominant negative mutants, which may be constructed by genetic engineering. They show no enzymatic activity but they compete for the respective substrate of the protein or enzyme that shall be inhibited, which results in a decrease in the proteins' activity. Another example for antagonists are antisense-molecules which can decrease the expression of a certain protein in a well known way. Agonists may be substances, which promote the expression of a certain gene or the translation of mRNA into the active gene product. This may be specific transcription factors or similar compounds that regulate the level of expression of the mentioned proteins. Especially small molecular compounds may be advantageously used for this purpose.

In a particularly preferred embodiment of the invention the active substance can be a therapeutic antibody which, as an inhibiting antibody can reduce or block the activity of the given protein preferably annexin A3. The therapeutic antibody can also be characterized in that it e.g. carries a toxic or radioactive label and by merely interacting with e.g. annexin A3 brings said label up to the cancer cells. This can e.g. be used during radioimmunotherapy the antibody carrying a radioactive label, e.g. ¹³¹I.

According to the invention the active substance can be a small molecular compound having a molecular weight (MW)<1000 for inhibiting the ion channel activity in membranes, preferably exosomes and/or matrix vesicle.

In order to increase the activity of the proteins described, especially of isopeptidase T, FABP-3, galectin-1, microseminoprotein beta, HSP27 and transgelin, a compound may be used that possesses a comparable or similar enzymatic activity. Furthermore the activity of the existing enzyme molecules may be induced or increased by a respective compound. On the other hand it is possible to use active substances that are suitable for induction or increase of the expression—that means the synthesis of the respective enzymatic molecules. The active substance may also address definite precursor molecules, regulators, activators or inhibitors of enzymes or other proteins.

Furthermore hormones or substances with similar effect may be used as active substances if they influence the activity of the respective protein in the desired way. For example molecules analogous to progesterone may be used to inhibit enoyl-coenzyme A hydratase.

In a special preferred embodiment of the application of the invention the active substance is at least one of the proteins itself: ubiquitin-isopeptidase T, serum-amyloid P-component (SAP), fatty acid-binding protein 3 (FABP-3), galectin-1, microseminoprotein beta, heat shock protein 27 (HSP27) and/or transgelin. The inventors could demonstrate that the abundance of these proteins is lowered in cancerous tissue therefore it is intended according to the invention to use the proteins themselves as active substances for the stimulation of their activity and/or expression. For this purpose single proteins or preferably a combination of several different proteins can be used. Furthermore it is intended that parts of these proteins, e.g. peptides or molecules derived from the proteins may be used as active substances according to the invention.

In a special preferred embodiment of the application of the invention one or more effective substances will be delivered as exosomes or the application of the active substance will be mediated by exosomes. This may preferably influence the patient's immune response, especially by modulating the T-cell response. Exosomes are membrane-coated vesicles that are secrete preferentially by haematopoietic cell. It is well known that exosomes produced by dendritic cell stimulate an effective anti-tumour response e.g. in mice.

By application of an active substance for the treatment of cancer according to the invention, advantageously a decrease or inhibition of development or growth of a tumour can be achieved and/or metastasis of tumours will be partly reduced or completely avoided.

Furthermore the invention covers a pharmaceutical composition, which contains at least one of the active substances described above and at least one pharmaceutically acceptable carrier. For details concerning the pharmaceutical composition or the active substance we refer to the description above. Suitable pharmaceutically acceptable carriers are clear to the experts.

Furthermore the invention covers a method for therapy of cancerous diseases e.g. prostate cancer by application of at least one of the described active substances. For further details of this method for cancer treatment we refer to the description above.

Different ways of administration may be used for the active substances administered, e.g. oral, intravenous, topic or administration via inhalation. Respective formulations are well known to the expert. The way of administration depends on the disease that shall be treated and of course on the patient's constitution. Details are familiar to the expert.

Finally the invention covers a method to search for active substances for cancer treatment, especially prostate cancer. With this method at least one protein will be used that may be chosen from the following groups: ubiquitin-isopeptidase T, serum-amyloid P-component (SAP), fatty acid-binding protein 3 (FABP-3), galectin-1, microseminoprotein beta, heat shock protein 27 (HSP27), 14-3-3 protein beta, 14-3-3 protein zeta, nuclear chloride ion channel protein 1(CLIC-1), 14-3-3 protein tau, heat shock protein 90 (HSP 90), protein-disulphide-isomerase (PDI), epidermal fatty acid-binding protein (E-FAPB), mitochondrial enoyl-coenzyme A hydratase, nucleophosmin, annexin, especially annexin A3, transgelin, triosephosphate-isomerase, aldolase A, HES 1, alpha 2-subunit of the proteaesome, adenine-phosphoribosyl-transferase and inorganic pyrophosphatase 1. Preferably advantageous are isopeptidase T, serum-amyloid P-component (SAP), nuclear chloride ion channel protein 1 (CLIC-1), 14-3-3 protein tau, mitochondrial enoyl-coenzyme A hydratase and/or annexin A3. Furthermore derivatives of these proteins can be used, especially homologous sequences or mutated forms of the proteins, which have been produced by methods of molecular biology. Furthermore parts of these proteins or subregions respectively or combinations of various proteins and/or their derivatives can be used. The implementation of the method is familiar to the expert, e.g. a protein or its derivative can be expressed with a suitable expression system. With the help of this system interactions with potential ligands, especially inhibitors or activators may be investigated. For example the two-hybrid-system is suitable for the investigation of these interactions.

The described characteristics and further features of the invention become clear from the following description of preferred embodiments in connection to subclaims and figures. Single characteristics may be realized alone or in combination with each other.

### EXAMPLES

In order to identify proteins relevant according to the invention the tissue samples of two patient groups (group A: 23 patients and group B: 33 patients) were examined. Cancerous tissue and control tissue were in each case prepared and subjected to two-dimensional polyacrylamide gel electrophoresis (2D-PAGE). Isoelectric focussing took place at pH 4-7 and pH 6-11. The gel electrophoretic results of two patients from group A were unsuitable for further analysis. In the case of a further two patients the results were unsatisfactory at pH 6-11. Thus, it was possible to evaluate the results of 21 patients in the pH range 4-7 and 19 patients in the pH range 6-11. The results of two patients from group B were unsuitable for further analysis at pH 4-7. Thus, in all the results of 31 patients in the pH range 4-7 could be evaluated.

The two different samples of each patient were labelled with in each case different isotopes mixed and electrophoretically separated on a single two-dimensional polyacrylamide gel. The signals of each isotope were then detected separately of one another, so that the protein samples of the two tissue samples could be directly compared (ProteoTope-technology).

For final identification of the proteins analytical amounts (<1 μg) of the radioactively labelled sample, together with preparative amounts (>200 μg) of non-labelled proteins of the same sample were separated in preparative gels. Relevant protein spots were cut out of silver stained preparation gels, enzymatically digested with trypsin and identified by matrix assisted laser desorption ionization time of flight mass spectrometry (MALDI-TOF MS) on a Bruker BiFlex or Ultra-Flex. Partly, electro spray ionization ion trap mass spectrometry (ESI-MS) was performed (Bruker Esquire).

In this way a variety of proteins were identified, which showed specifically a corresponding upregulation or downregulation of abundance in cancerous tissue as compared to control tissue.

For these analyses, in part specific patient groups were formed within which the abundances of different proteins were investigated. In this so-called cluster analysis (Clustan Graphics 6.4) three groups of patients from group A and two groups of patients from group B were determined which in each case revealed characteristic protein expression/abundance patterns. With the aid of this procedure identification took place of the proteins annexin A3, transgelin, triosephosphate-isomerase and aldolase A, which for specific patients revealed characteristic abundances.

Tissue Samples

Healthy prostate tissue and malignant prostate tissue were received from patients after prostectomy. The patients were screened for PSA (prostate specific antigen) and the tumours were confirmed by ultrasonic scans. Consent of every patient was received before surgery.

Immediately after removal, the prostate gland was transferred to a sterile box and cooled. Tissue slices of 0.5-1 cm thickness were prepared and divided into left and right half. These were embedded into a freezing matrix and shock frozen. The remains of the prostate gland were fixed in formaldehyde solution and further treated according to common standard methods. For the preparation of tissue samples thin sections of both sides of the prostate gland were taken and stained with Haematoxilin-Eosin. These sections were stored at −80° C. until they were used. Control tissue samples were taken from tumour-free regions and treated identically.

Sample Preparation

Proteins were lysed with 100 μl of a boiling solution containing 2% SDS, 0.1 M Tris pH 8.8 and protein concentration was determined with the bicinchoninic acid-method.

Iodination with ¹²⁵I or ¹³¹I respectively, two-dimensional polyacrylamide gel electrophoresis and data analysis were performed according to common protocols (Cahill et al., 2003 Rapid Communications in Mass Spectrometry 17: 1283-1290). Radioactive iodine was purchased from Amersham Bioscience (Freiburg, Germany). Iodination reactions were performed separately with equal concentrations of either ¹²⁵I or ¹³¹I.

Polyacrylamide Gel Electrophoresis

For application on the polyacrylamide gels identical amounts of proteins of the labelled samples (cancer tissue and control tissue) were mixed. For isoelectric focussing (IEF) in the ranges of pH 4-7 and 6-11 samples were diluted in a common sample buffer and loaded on 18 cm pH-gradient (IPG) stripes (Amersham Bioscience). IEF as the first dimension of separation of proteins by 2D-PAGE was performed on a Multiphor apparatus (Amersham Bioscience). The second dimension (SDS-PAGE) was performed on an ISO-DALT apparatus (Höfer). Gels were dried, laminated on an 80 μm plastic film and finally measurement of the signals of the two radioactive isotopes was carried out.

With the selected method for analysis of the different radioisotopes a quantitative multicoloured differential display of the proteins from the different sample could be achieved. Therefore a direct comparison of the integrated intensities of the protein spots separated on one gel could be used for further analysis. Analysis on a single gel offers the advantage that systematic errors in variations among two or more gels become irrelevant. The largest potential source for errors is a different stoichiometry in labelling with either isotope. This could be excluded by performing gels with reverse labelling, i.e. control sample and cancer ample were each labelled with either isotope and compared inversely. Patterns of protein expression for the inverse labelling procedures were matching, therefore quantitative criteria were fulfilled. By means of computer-aided methods the signals for the different isotopes were visualized in different colour (blue or orange) consequently consistent differences in abundance among the samples were displayed in one of the two colours corresponding to the isotope which was used for labelling. Details concerning this methodology are mentioned in Cahill et al., 2003 Rapid Communications in Mass Spectrometry 17: 1283-1290.

Image Analysis

Differential analysis of protein expression is based on a reliable differential quantification of protein spots in polyacrylamide-gels described. For quantitative image analysis the software Phoretix 2D Advanced (Nonlinear Dynamics) was used with special adaptations made by the inventors.

Identification of Proteins By Mass Spectrometry

In principle two different methods of mass spectrometry were used. On the one hand the fast and reliable identification of high abundance proteins via peptide mass fingerprinting with MALDI-TOF MS. Identification of very low abundant proteins was performed using the more time consuming LC-ESI-Ion-Trap-MS/MS or MALDI-TOF-TOF procedures. In summary pieces of gels containing the selected protein spots were cut off and the proteins in the gel pieces were digested with trypsin. First the resulting solution was analyzed by peptide mass fingerprinting with MALDI-TOF-MS. For protein spots which could not be identified unambiguously by doing so the slower fragmentation analysis basing on MALDI-TOF-TOF or LC-ESI-Ion-Trap-MS/MS was performed. A detailed description of these methods can be found in: Vogt et al., 2003, Molecular Cellular Proteomics 2: 795.

Identification of Proteins

For identification of the proteins their peptide masses which had been found by mass spectrometry were analyzed using the NCBI-database. This was done with the program MASCOT version 1.9 (Matrix Science, London, UK).

Quantitative image Analysis

Quantitative analyses were performed using digital data that had been recorded by the photomultiplier of a radio-imager for every pixel of the picture matrix. Limits of the protein spots were defined with the help of the software Phoretix 2D Advanced (Nonlinear Dynamics) and the pixel results inside the spot region were integrated after subtraction of a suitable background signal. Based on the complete data that were generated, a detailed quantification of the detected protein spots was performed. Table 1 summarizes these results.

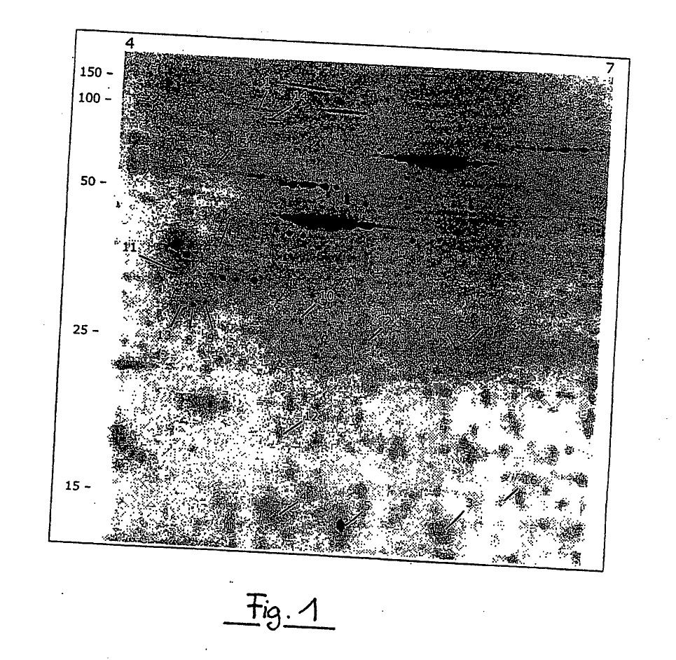

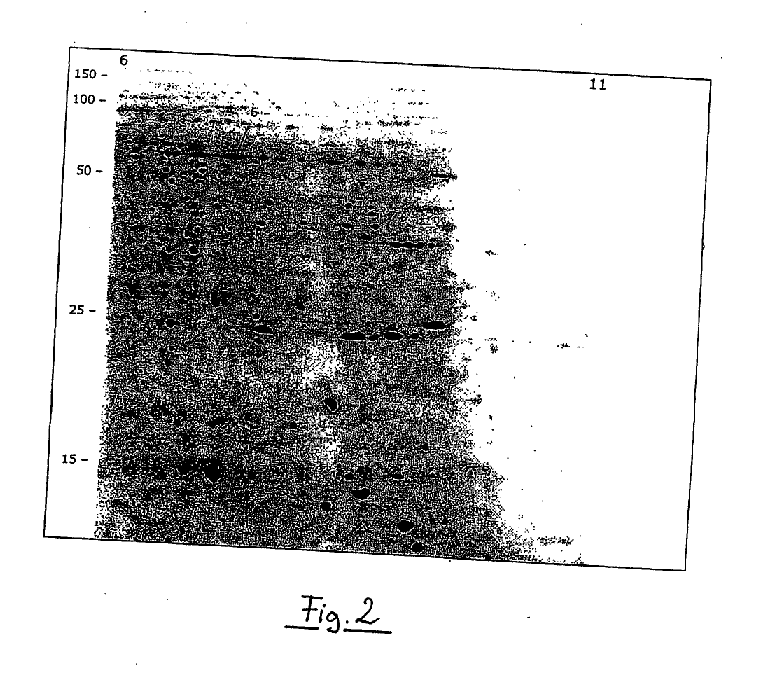

FIG. 1 and FIG. 2 respectively show the positions of selected spots after isbelectric focussing at pH 4-7 (FIG. 1) and in the second case at pH 6-11 (FIG. 2).

Figures Legends:

FIG. 1: Image of a two-dimensional polyacrylamide gel with separated proteins. Isoelectric focussing was performed at pH 4-7. The protein spots labelled by numbers show those proteins whose abundance differs in cancerous tissue and control tissue respectively. The numbers refer to those in table 1.

FIG. 2: Representation of a two-dimensional polyacrylamide gel with separated proteins. Isoelectric focussing was performed at pH 6-11. The protein spots labelled by numbers show those proteins whose abundance differs in cancerous tissue and control tissue respectively. The numbers refer to those in table 1.

FIG. 3: Graphical representation of patterns of protein expression that are characteristic for distinct patient collectives, i.e. distinct subtypes of prostate cancer. Results, which are statistically significant with p<0.01 are drawn in black colour, results which t-test p-values of 0.01<p<0.1 are drawn in grey colour. Proteins with varying expression within the different clusters are shown in frames.

FIG. 4: Tabular representation of the different levels of protein expression in patient collectives 1 to 3 comparing benign (healthy). The data refer to percentage of the protein spot's size in cancerous tissue with standard error in relation to the total volume of the protein spots (benign+malignant). The t-test probability represents the likelihood that the distribution of the spot fraction of two given collectives is significantly different. T-test results of more than 99% probability are printed in bold. Results with probabilities lower than 95% are printed in light grey.

FIG. 5: The table lists protein spots with significant differential expression in all patients comparing benign (healthy) and malignant tissue, based on a two-colour ProteoTope analysis: “No. Obs. represents the number of patients where the spot could be observed. The spot-fraction for benign tissue (benign fraction) and malignant tissue (cancer fraction) with standard error refers to the percentages of the total volume of the protein spots (benign+malignant). The t-test probability represents the likelihood that the distribution of the spot fraction in benign tissue differs significantly from the distribution in cancer tissue, when all patients are taken into account. Spots were selected under the condition that the t-test probability amounted to at least 99%.

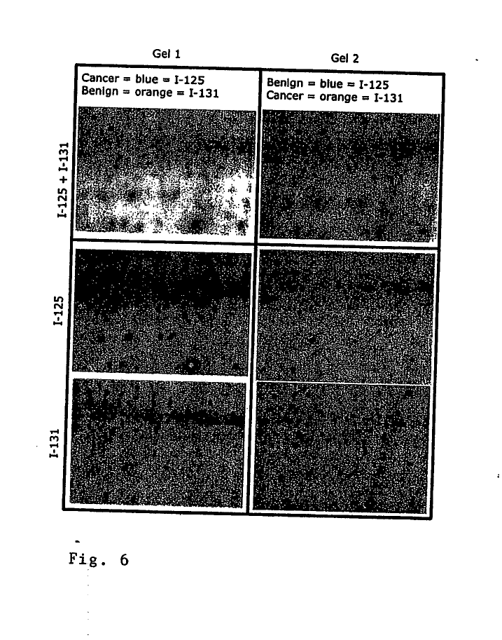

FIG. 6: Presentation of a two-dimensional polyacrylamide gel with separated proteins of patient 14 from group B. Both the control sample and the cancerous tissue sample were labelled with ¹³¹I and ¹²⁵I and inversely compared. The different isotope signals are in each case rendered visible in another colour (blue/orange), so that there are consistent differences in the abundance of proteins between the samples in each of the colours, as a function of the chosen isotope labelling.

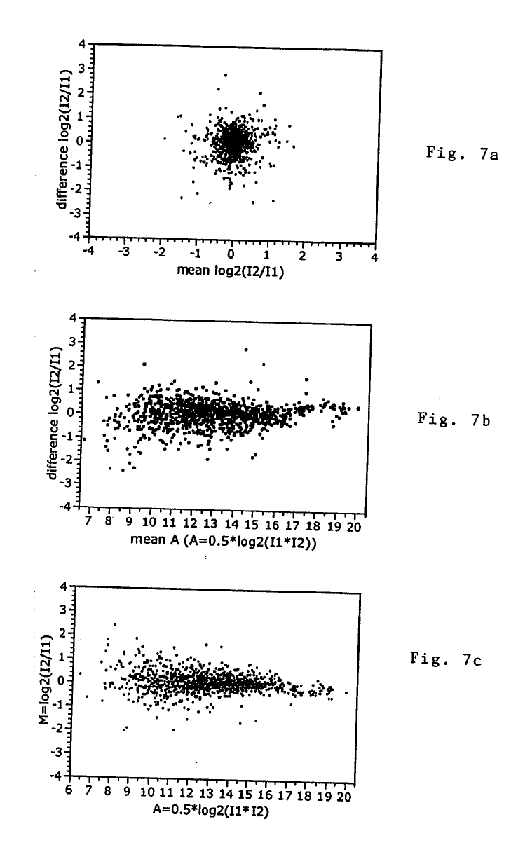

FIG. 7: Graphs representing the precision and statistical significance of the Proteo-Tope measurements using the example of group B:
a. Bland and Altman Plot reproducing the ratio between the difference in the differential abundance ratio M and its arithmetic mean for a gel labelled with ¹²⁵I and ¹³¹I,
b. Plot reproducing the ratio between the difference in the differential abundance ratio M and the arithmetic mean of the intensity A for a gel labelled with ¹²⁵I and ¹³¹I,
c. MA Plot reproducing the ratio between the differential abundance ratio M and the intensity A for a gel labelled with ¹²⁵I and ¹³¹I, in which M=log 2·(I2/I1) and A=0.5·log 2·(I1·I2) (I=measured intensity).

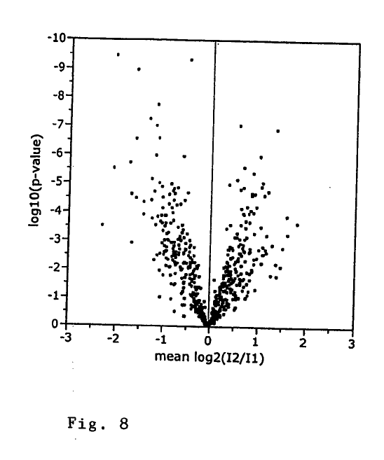

FIG. 8: Volcano Plot showing the difference between the average intensities of the detected inversely labelled proteins from cancerous and healthy tissue.

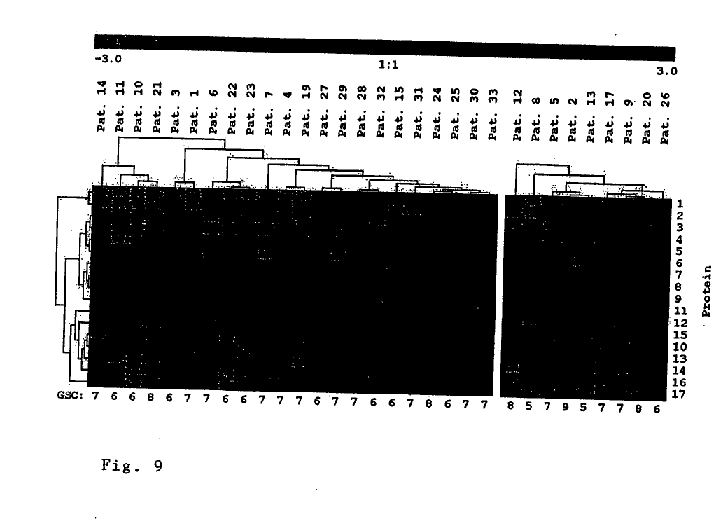

FIG. 9: Graph of Pavlidis Template Matching Analysis which, in the case of the cancer patients from group B, provides two subgroups of protein abundance ratio patterns. One group consists of 22 patients, whilst the other which differs significantly therefrom consists of 9 patients. The protein numbers correspond to the numbers in the table of FIG. 10. Within the subgroup of 22 patients there is a significant difference in the relative abundance of annexin A3 (protein 14). In the case of patients 14 11, 10, 21, 3, 1, 6, 22, 23, 7, 4, 19 and 27 the protein is much more abundant in malignant prostate tissue than in patients 29, 28, 32, 15, 31, 24, 25, 30 and 33.

FIG. 10: Table showing the protein spots from the differential analysis of all 31 patients (group B), the group with 22 and the group with 9 patients (obtained by Pavlidis Template Matching Analysis). The accession number corresponds to the number from the NCBI data bank. The scores are obtained using MASCOT technology. The indication relative to the PMF score relates to the mouse score used by the MASCOT server and in general a PMF score above 65 represents a significant identification. The identity of the proteins carrying an asterisk was determined by LC/MS/MS. The average spot fraction of cancerous tissue is given with standard errors as a percentage of the total spot volume (healthy+malignant). The P value for this model is also given. The balcony in the table gives the average abundance as a per cent of each protein in the benign (dark blue) and cancerous (light orange) samples in the indicated patient groups.

1. Use of the protein annexin A3 as a diagnostic marker for prostate cancer.

2. Use according to claim 1, characterized in that it is a matter of specific subtypes of prostate cancer.

3. Use according to claim 1 characterized in that an upregulation of annexin A3 compared with controls is investigated.

4. Use according to claim 1, characterized in that an upregulation of annexin A3 combined with a downregulation of annexin A1, annexin A2 and/or annexin A5 is investigated.

5. Use of at least one active substance which interacts with the protein annexin A3 and in particular influences, preferably inhibits the activity and/or the abundance of the protein annexin A3, for producing a medicament for the treatment of prostate cancer, preferably specific prostate cancer patient groups.

6. Use according to claim 5, characterized in that the active substance is an agonist, antagonist, a deficient mutant, a dominant negative mutant and/or an antisense molecule.

7. Use according to claim 5, characterized in that the active substance is an antibody, preferably a therapeutic antibody.

8. Use according to claim 5, characterized in that the active substance is at least one benzodiazepine derivative, particularly BDA250 and/or BDA452.

9. Use according to claim 5, characterized in that the activity and/or abundance of the protein annexin A3 in exosomes is influenced.

10. Use according to claim 5, characterized in that the active substance is a small molecular compound with a molecular weight (MW)<1000 for inhibiting the ion channel activity in membranes, preferably exosomes and/or matrix vesicles.

11. Use of the protein mitochondrial enoyl-coenzyme A-hydratase as a diagnostic marker for cancer.

12. Use according to claim 11, characterized in that an upregulation of mitochondrial enoyl-coenzyme A-hydratase compared with controls is investigated.

13. Use of the protein ubiquitin-isopeptidase T and/or protein-disulphide-isomerase (PDI) as a diagnostic marker for cancer.

14. Use according to claim 13, characterized in that a downregulation of ubiquitin-isopeptidase T and/or an upregulation of protein-disulphide-isomerase (PDI) compared with controls is investigated.

15. Use of the protein serum-amyloid P-component (SAP) as a diagnostic marker for cancer.

16. Use according to claim 15, characterized in that a downregulation of serum-amyloid P-component (SAP) compared with controls is investigated.

17. Use of the protein nuclear chloride ion channel protein as a diagnostic marker for prostate cancer.

18. Use according to claim 17, characterized in that an upregulation of the nuclear chloride ion channel protein is investigated when compared with controls.

19. Use of the protein HES1 as a diagnostic marker for cancer.

20. Use according to claim 19, characterized in that an upregulation of HES1 compared with controls is investigated.

21. Use of the proteasome alpha 2-subunit as a diagnostic marker for cancer.

22. Use according to claim 21, characterized in that an upregulation of the proteasome alpha 2-subunit compared with controls is investigated.

23. Use of the protein adenine-phosphoribosyl-transferase as a diagnostic marker for prostate cancer.

24. Use according to claim 23, characterized in that an upregulation of the adenine-phosphoribosyl-transferase compared with controls is investigated.

25. Use of the protein inorganic pyrophosphatase as a diagnostic marker for prostate cancer.

26. Use according to claim 25, characterized in that an upregulation of inorganic pyrophosphatase compared with controls is investigated.

27. Use of the proteins ubiquitin-isopeptidase T and serum-amyloid P-component (SAP) as diagnostic markers for cancer, in which preferably a downregulation of the proteins compared with controls is investigated.

28. Use of at least two proteins selected from the group consisting of ubiquitin-isopeptidase T, heat shock protein 27 (HSP27), heat shock protein 90 (HSP90), protein-disulphide-isomerase (PDI), mitochondrial enoyl-coenzyme A-hydratase and nucleophosmine as diagnostic markers for cancer, in which there is an investigation of a downregulation of ubiquitin-isopeptidase T and/or heat shock protein 27 (HSP27) and/or an upregulation of heat shock protein 90 (HSP90), protein-disulphide-isomerase (PDI), mitochondrial enoyl-coenzyme A-hydratase and/or nucleophosmine compared with controls.

29. Use according to claim 1, characterized in that the cancer is prostate cancer.

30. Use of claim 1, characterized in that through the investigation of one or more proteins subtypes of cancer, particularly prostate cancer are diagnosed.

31. Use according to the protein annexin A3 as a diagnostic marker for prostate cancer, characterized in that through the investigation of one or more proteins subtvpes of cancer, particularly prostate cancer are diagnosed, characterized in that at least one protein according to claim 28 in combination with at least one protein selected from the group consisting of serum-amyloid P component (SAP), fatty acid-binding protein 3 (FABP-3), galectin, microseminoprotein beta, 14-3-3 protein beta, 14-3-3 protein zeta, nuclear chloride ion channel protein, 14-3-3 protein tau, epidermal fatty acid-binding protein (E-FABP), annexin A3, transgelin, triosephosphate isomerase and aldolase A are investigated, an investigation taking place of zero or minor modifications of SAP, a downregulation of FABP-3, a strong downregulation of galectin, a strong downregulation of microseminoprotein beta, zero or minor changes of 14-3-3 protein beta, zero or minor changes of 14-3-3 protein zeta, zero or minor changes of nuclear chloride ion channel protein, zero or minor changes of 14-3-3 protein tau, zero or minor changes of E-FABP, zero or minor changes of annexin A3, an upregulation of transgelin, zero or minor changes of triosephosphate isomerase and/or zero or minor changes of aldolase A compared with controls.

32. Use of the protein annexin A3 as a diagnostic marker for prostate cancer, characterized in that through the investigation of one or more proteins subtvpes of cancer, particularly prostate cancer are diagnosed, characterized in that at least one protein according to claim 28 in combination with at least one protein selected from the group consisting of serum-amyloid P component (SAP), fatty acid-binding protein 3 (FABP-3), galectin, microseminoprotein beta, 14-3-3 protein beta, 14-3-3 protein zeta, nuclear chloride ion channel protein, 14-3-3 protein tau, annexin A3, transgelin, triosephosphate-isomerase and aldolase A are investigated, investigation taking place of a strong upregulation of PDI, a strong upregulation of HSP90, a strong downregulation of ubiquitin-isopeptidase T, a downregulation of SAP, zero or minor changes of FABP-3, a downregulation of galectin, a downregulation of microseminoprotein beta, an upregulation of 14-3-3 protein beta, an upregulation of 14-3-3 protein zeta, an upregulation of 14-3-3 protein tau, zero or minor changes of nuclear chloride ion channel protein, an upregulation of annexin A3, a downregulation of transgelin, an upregulation of triosephosphate isomerase and/or an upregulation of aldolase A compared with controls.

33. Use of the protein annexin A3 as a diagnostic marker for prostate cancer, characterized in that through the investigation of one or more proteins subtvpes of cancer, particularly prostate cancer are diagnosed, characterized in that at least one protein according to claim 28 in combination with at least one protein selected from the group consisting of serum-amyloid P component (SAP), fatty acid-binding protein 3 (FABP-3), galectin, microseminoprotein beta, 14-3-3 protein beta, 14-3-3 protein zeta, nuclear chloride ion channel protein, 14-3-3 protein tau, epidermal fatty acid-binding protein (E-FABP), annexin A3, transgelin, triosephosphate-isomerase and aldolase A are investigated, an investigation taking place of a downregulation of SAP, zero or minor changes of FABP-3, zero or minor changes of galectin, zero or minor changes of microseminoprotein beta, zero or minor changes of 14-3-3 protein beta, zero or minor changes of 14-3-3 protein zeta, a strong upregulation of nuclear chloride ion channel protein, zero or minor changes of 14-3-3 protein tau, zero or minor changes of E-FABP, zero or minor changes to annexin A3, zero or minor changes of transgelin, zero or minor changes of triosephosphate-isomerase and/or zero or minor changes of aldolase A compared with controls.

34. Use according to claim 1, characterized in that at least one protein is detected with the aid o polyacrylamide gel electrophoresis, particularly two-dimensional gel electrophoresis, mass spectrometry, positron-radiation tomography (PRT), antibodies, ELISA, immunohistochemistry, protein chips and/or oligonucleotides, particularly the polymerase chain reaction (PCR).

35. Use according to claim 1, characterized in that exosomes are isolated and/or analyzed for investigating the at least one protein.

36. Diagnostic kit, comprising at least one substance for detecting the activity and/or abundance of at least one protein selected from the group consisting of ubiquitin-isopeptidase T, serum-amyloid P component (SAP), nuclear chloride ion channel protein, mitochondrial enoyl-coenzyme A-hydratase and annexin A3 for the identification of cancerous diseases, particularly prostate cancer.

37. Use of at least one active substance influencing the activity and/or abundance of the proteins ubiquitin-isopeptidase T and protein-disulphide-isomerase (PDI), for producing a medicament for the treatment of cancer, in which preferably the active substance increases the activity and/or abundance of ubiquitin-isopeptidase T and/or the active substance inhibits the activity and/or abundance of the protein-disulphide-isomerase (PDI).

38. Use of at least one active substance influencing the activity and/or abundance of the protein mitochondrial enoyl-coenzyme A-hydratase for producing a medicament for the treatment of cancer.

39. Use according to claim 38, characterized in that the active substance inhibits the activity and/or abundance of the mitochondrial enoyl-coenzyme hydratase.

40. Use of at least one active substance influencing and in particular increasing the activity, abundance and/or localization of the protein serum-amyloid P-component (SAP) for producing a medicament for the treatment of cancer.

41. Use of at least one active substance influencing, particularly inhibiting, the activity and/or abundance of the protein nuclear chloride ion channel protein for producing a medicament for the treatment of prostate cancer.

42. Use of at least one active substance influencing, particularly inhibiting, the activity and/or abundance of protein HES1 for producing a medicament for the treatment of cancer.

43. Use of at least one active substance influencing, particularly inhibiting, the activity and/or abundance of the proteasome alpha 2-subunit for producing a medicament for the treatment of cancer.

44. Use of at least one active substance influencing, particularly inhibiting, the activity and/or abundance of the protein adenine-phosphoribosyl transferase for producing a medicament for the treatment of prostate cancer.

45. Use of at least one active substance influencing, particularly inhibiting, the activity and/or abundance of the protein inorganic pyrophosphatase for producing a medicament for the treatment of prostate cancer.

46. Use according to claim 37, characterized in that the cancer is prostate cancer, preferably specific prostate cancer subtypes.

47. Use according to claim 37, characterized in that the active substance is an agonist, antagonist, a deficient mutant, a dominant-negative mutant and/or an antisense molecule.

48. Use according to claim 37, characterized in that the active substance is an antibody, preferably a therapeutic antibody.

49. Use according to claim 37, characterized in that the active substance is a small molecular compound with a molecular weight (MW)<1000 for inhibiting ion channel activity in membranes, preferably exosomes and/or matrix vesicles.

50. Use according to claim 1, characterized in that the active substance is at least one protein selected from the group of ubiquitin-isopeptidase T, serum-amyloid P-component (SAP), fatty acid-binding protein 3 (FABP-3), annexin A3, galectin, microseminoprotein beta, heat shock protein 27 (HSP27) and transgelin.

51. Use according to claim 1, characterized in that the active substance is provided in the form of exosomes.

52. Pharmaceutical composition comprising at least one active substance according to claim 1, and at least one pharmaceutically acceptable carrier.

53. Method for seeking active substances for the treatment of cancer, characterized in that at least one protein selected from the group consisting of ubiquitin-isopeptidase T, serum-amyloid P-component (SAP), nuclear chloride ion channel protein, 14-3-3 protein tau, mitochondrial enoyl-coenzyme A-hydratase, annexin A3, HES1, proteasome alpha 2-subunit, adenine-phosphoribosyl transferase and inorganic pyrophosphatase and/or at least one derivative thereof is used.
